(19) United States
(12) Patent Application Publication
(10) Pub. No.: US 2026/0051061 A1
(43) Pub. Date: Feb. 19, 2026

(54) METHOD FOR NON-INVASIVE DETECTION OF PRESSURE INJURIES OF SKIN

(71) Applicant: WEST CHINA HOSPITAL, SICHUAN UNIVERSITY, CHENGDU (CN)

(72) Inventors: YAN JIANG, CHENGDU (CN); KE XU, CHENGDU (CN); HUA CAO, CHENGDU (CN)

(21) Appl. No.: 19/290,632

(22) Filed: Aug. 5, 2025

(30) Foreign Application Priority Data
Aug. 15, 2024 (CN) 202411116684.1

(51) Int. Cl. G06T 7/00; A61B 5/00

(52) U.S. Cl. CPC G06T 7/0014; A61B 5/445; G06T 2207/10016; G06T 2207/20024; G06T 2207/20056; G06T 2207/20076; G06T 2207/20172; G06T 2207/30088; G06T 2207/30096

(57) ABSTRACT

The invention relates to the technical field of image processing, in particular to a method for non-invasive detection of pressure injuries of skin. The method includes: extracting a skin video image and transforming the skin video image to a YIQ color space; magnifying the preprocessed skin video image based on a Eulerian video magnification algorithm to extract skin luminance signals; performing correlation analysis on the skin luminance signals to recognize a skin region with potential pressure injuries; and respectively calculating, by means of a transfer function, a power spectral density of the skin region with the potential pressure injuries and a power spectral density of a normal skin region to obtain a detection result. The recognition accuracy and efficiency of pressure sores are improved.

### BACKGROUND OF THE INVENTION

### 1. Technical Field

The invention relates to the technical field of image processing, in particular to a method for non-invasive detection of pressure injuries of skin.

### 2. Description of Related Art

Pressure sores, medically known as pressure injuries, are a pathological state of cell dysfunction and even tissue necrosis caused by restriction on blood circulation resulting from a continuous local pressure or a pressure and a shear force applied to skin and even subcutaneous tissue. Traditional clinical detection for pressure sores mainly relies on direct observation and tactile sensation of medical staff, that is, medical staff determine the presence of pressure sores and the severity of the pressure sores by observing the change in color of skin and the swelling of skin and sensing the hardness of skin by hand. Such a detection method highly relies on professional knowledge, experience and intuitive judgment of medical staff, have some limitations and is low in detection efficiency.

With the development of the image recognition technology, professional researchers in the skin detection field are seeking for a novel detection method combined with the image recognition technology to overcome limitations and low efficiency in pressure sore detection. In the prior art, by means of an integrated deep learning model and a computer vision algorithm, a system may automatically recognize thermal images related to pressure injuries from high-resolution images to assist in diagnosis of pressure sores to improve the detection efficiency of the pressure sores.

However, in clinical practice, early recognition of pressure injuries is of particular importance. Because the pressure injuries are merely manifested by a minor change in skin color or a tiny difference in tissue in the initial stage, and these changes cannot be easily recognized by an existing image recognition method and may be ignored or misjudged under a complex background, leading to low recognition accuracy. In view of this, a method for non-invasive detection of pressure injuries of skin is proposed.

### BRIEF SUMMARY OF THE INVENTION

To improve the recognition accuracy and efficiency of pressure sores, the objective of the invention is to provide a method for non-invasive detection of pressure injuries of skin. The technical solution adopted by the invention is specifically as follows:

A method for non-invasive detection of pressure injuries of skin includes:
a step of extracting a skin video image and transforming the skin video image to a YIQ color space;
a step of magnifying the preprocessed skin video image based on a Eulerian video magnification algorithm to extract skin luminance signals;
a step of performing correlation analysis on the skin luminance signals to recognize a skin region with potential pressure injuries; and
a step of respectively calculating, by means of a transfer function, a power spectral density of the skin region with the potential pressure injuries and a power spectral density of a normal skin region to obtain a detection result.

Further, the step of magnifying the preprocessed skin video image based on a Eulerian video magnification algorithm to extract skin luminance signals includes:
a step of constructing an image downsampling pyramid and extracting a high-level skin image by weighted averaging of adjacent pixels;
a step of constructing an image upsampling pyramid and restoring the high-level skin image to an original resolution; and
a step of extracting luminance signals of normal skin and luminance signals of skin with pressure injuries from a skin image obtained by upsampling.

Further, the step of constructing an image downsampling pyramid and extracting a high-level skin image by weighted averaging of adjacent pixels includes:
selecting coordinates of a central pixel of the skin image corresponding to a target layer of the downsampling pyramid and scaling the coordinates to obtain a corresponding pixel position in the skin image corresponding to a layer below the target layer of the downsampling pyramid; and
taking into account pixels within a preset variation range around the central pixel of the skin image corresponding to the target layer, performing weighted summation to obtain pixels at corresponding pixel positions in the skin image corresponding to the layer below the target layer.

Further, the step of constructing an image upsampling pyramid and restoring the high-level skin image to an original resolution includes:
a step of performing scaling based on pixel positions of a skin image obtained by downsampling to obtain pixel positions in the skin image corresponding to a target layer of the upsampling pyramid; and
a step of taking into account pixels within a preset variation range around the central pixel of the skin image corresponding to the target layer of the upsampling pyramid, performing weighted summation to obtain pixels at the corresponding pixel positions in the skin image corresponding to the target layer of the upsampling pyramid.

Further, the step of magnifying the preprocessed skin video image based on a Eulerian video magnification algorithm to extract skin luminance signals further includes:
a step of separating out, by a band-pass filter, frequency components related to the skin with the pressure injuries from the skin image obtained by downsampling according to a preset frequency; and
a step of performing spatial filtering and amplification on signals output by the band-pass filter and extracting the luminance signals of the normal skin and the luminance signals of the skin with the pressure injuries.

Further, the step of performing correlation analysis on the skin luminance signals to recognize a skin region with potential pressure injuries includes:
a step of performing Z-score standardization on the luminance signals of the normal skin and the luminance signals of the skin with the pressure injuries; and
a step of performing covariance analysis on the luminance signals of the normal skin and the luminance signals of the skin with the pressure injuries to obtain a correlation coefficient and a covariance of the luminance signals of the normal skin and the luminance signals of the skin with the pressure injuries.

Further, the step of performing correlation analysis on the skin luminance signals to recognize a skin region with potential pressure injuries includes:
a step of normalizing the luminance signals of the normal skin and luminance signals of the skin with the pressure injuries; and
a step of performing differential analysis on the normalized luminance signals of the normal skin and the normalized luminance signals of the skin with the pressure injuries.

Further, the step of performing differential analysis on the normalized luminance signals of the normal skin and the normalized luminance signals of the skin with the pressure injuries includes:
a step of calculating a root-mean-square error of the luminance signals of the normal skin and a root-mean-square error of the luminance signals of the skin with the pressure injuries; and
a step of performing fast Fourier transform on the normalized luminance signals of the normal skin and the normalized luminance signals of the skin with the pressure injuries and extracting signals processed by fast Fourier transform.

Further, the step of respectively calculating, by means of a transfer function, a power spectral density of the skin region with the potential pressure injuries and a power spectral density of a normal skin region to obtain a detection result includes:
a step of calculating the power spectral densities based on the signals processed by fast Fourier transform; and
a step of dividing the power spectral density of the normal skin region by the power spectral density of the skin region with the potential pressure injuries to obtain a correlation graph and outputting the detection result.

Further, the method further includes: a step of extracting, based on empirical mode decomposition, intrinsic mode functions from the skin region with the potential pressure injuries.

The invention has the following beneficial effects:

According to the method for non-invasive detection of pressure injuries of skin, the skin video image is extracted and transformed to the YIQ color space, and in the YIQ color space, luminance information is separated from hue and saturation information, such that more attention is paid to changes of skin luminance signals in the skin image processing process; then, the tiny changes of the skin luminance signals are enhanced by means of the Eulerian video magnification algorithm, and by magnification, the tiny changes related to pressure injuries of skin may be observed more clearly; the correlation coefficient between the luminance signals of normal skin and the luminance signals of skin with pressure injuries is calculated, and a linear relation between the luminance signals of the normal skin and the luminance signals of the skin with the pressure injuries is evaluated, such that a region possibly with pressure injuries is preliminarily screened out; and finally, the power spectral density of the skin region with the potential pressure injuries and the power spectral density of the normal skin region are calculated respectively by means of the transfer function to further analyze nonlinear characteristics of the luminance signals of the skin region with the potential pressure injuries, a specific mode related to the pressure injuries of skin is recognized, and specific frequency components related to the pressure injuries of skin are recognized based on the power spectral densities, thus improving the detection accuracy and reliability of the pressure injuries of skin.

### BRIEF DESCRIPTION OF THE SEVERAL VIEWS OF THE DRAWINGS

To better clarify the technical solutions and advantages of the embodiments of the invention or the prior art, drawings used for describing the embodiments of the invention or the prior art are briefly introduced below. Obviously, the drawings in the following description are merely illustrative ones, and are not all possible ones of the invention. For those ordinarily skilled in the art, other drawings may be obtained according to the following ones without creative labor.

FIG. 1 is a schematic flow diagram of a method for non-invasive detection of pressure injuries of skin according to an embodiment of the invention.

FIG. 2 is a schematic flow diagram of extracting skin luminance signals according to an embodiment of the invention.

### DETAILED DESCRIPTION OF THE INVENTION

To further expound the technical means adopted by the invention to fulfill specific purposes and the effects of the invention, the specific implementation, structures, features and effects of a method for non-invasive detection of pressure injuries of skin provided by the invention are described in detail below in conjunction with drawings and preferred embodiments. In the following description, “one embodiment” or “another embodiment” in different positions does not definitely refer to the same embodiment. In addition, the specific features, structures or characteristics in one or more embodiments may be combined in any proper form.

Unless otherwise defined, all technical and scientific terms used here have the same meaning as commonly understood by those skilled in the technical field of the invention.

A specific scheme of the method for non-invasive detection of pressure injuries of skin provided by the invention is explained specifically below in conjunction with the drawings.

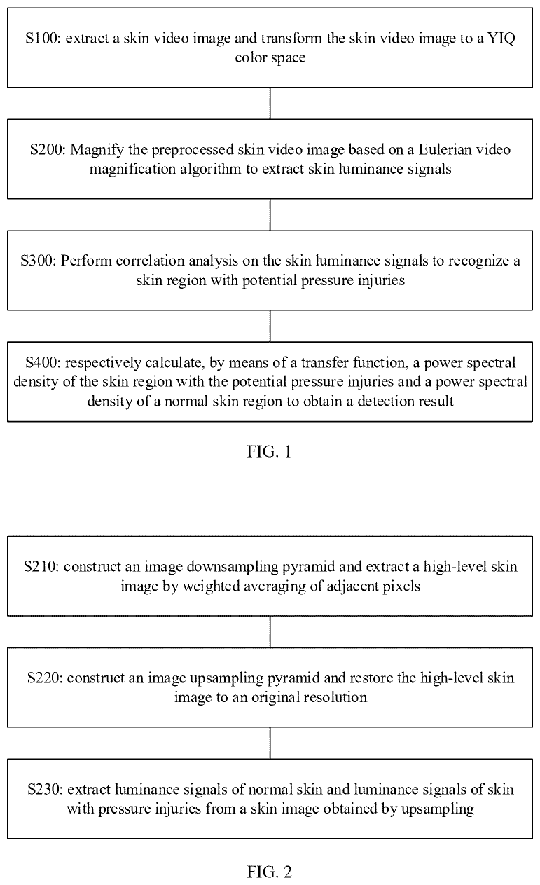

Referring to FIG. 1 which illustrates a schematic flow diagram of the method for non-invasive detection of pressure injuries of skin according to an embodiment of the invention, the method includes the following steps:

S100: a skin video image is extracted and transformed to a YIQ color space.

Specifically, a real-time video image of the skin of a patient is acquired by means of an image video sensor, a high-resolution camera is preferably used to acquire the skin image and a light source is used for auxiliary lighting to ensure that the acquired image is clear and lighting is uniform, and a display allowing for interactive operation may be used for previewing and adjusting the image acquisition angle and position to guarantee the accuracy of the acquired skin image; and acquired video information is transmitted to a PC host for image processing. The PC host performs preprocessing, which at least includes operations such as image denoising, luminance uniformization and signal amplification, on the acquired video image to improve the accuracy of subsequent processing. In this embodiment, specifically, in the YIQ color space, the skin image information is divided into a luminance channel (Y) and chrominance information (I and Q), a skin region is extracted from the video image, an image in an RGB color space is transformed to the YIQ color space, and the luminance channel contains luminance information of the skin image. In the YIQ color space, by merely magnifying a change of the luminance channel by a Eulerian video magnification algorithm later, tiny motions in the image may be enhanced without introducing a significant color distortion, thus improving the detection accuracy of pressure injuries of skin.

S200: the preprocessed skin video image is magnified based on a Eulerian video magnification algorithm to extract skin luminance signals. Specifically, tiny changes in the skin image transformed to the YIQ space may be magnified by the Eulerian video magnification algorithm, such that pressure injuries of skin may be observed more easily.

Referring to FIG. 2, S200 specifically includes:

S210: an image downsampling pyramid is constructed, and a high-level skin image is extracted by weighted averaging of adjacent pixels to reduce the resolution of the image and maintain major features of the image. By constructing the downsampling pyramid and extracting multi-layer representations of the skin image, multi-scale information is provided for further processing.

S210 specifically includes:

S211: coordinates of a central pixel of the skin image corresponding to a target layer of the downsampling pyramid are selected and scaled to obtain a corresponding pixel position in the skin image corresponding to a layer below the target layer of the image downsampling pyramid. Because the resolution of the image will decrease by half downwards every one layer in the downsampling pyramid, the coordinates of the skin image corresponding to an i^{th} layer needs to be scaled down by half to obtain corresponding pixel positions in the image corresponding to the next layer; and

S212: taking into account pixels within a preset variation range around the central pixel of the skin image corresponding to the target layer, weighted summation is performed to obtain pixels at corresponding pixel positions in the skin image corresponding to the layer below the target layer.

The downsampling pyramid is expressed as:

$${G}_{i + 1} ( x , y ) = {\sum }_{m = - 2}^{2} {\sum }_{n = - 2}^{2} w ( m , n ) {G}_{i} ( 2 x + m , 2 y + n ) \qquad ( 1 )$$

in formula (1), Gᵢ₊₁(x, y) denotes a pixel at coordinates (x, y) of the skin image corresponding to a (i+1)th layer of the downsampling pyramid, that is, the pixel obtained by downsampling of the skin image corresponding to the i^{th} layer of the downsampling pyramid; w(m, n) denotes a weight function, which determines the degree of influence on pixels in the original skin image on pixels in the new skin image during the downsampling process and is a generally a Gaussian weight function used for smoothening the image; Gᵢ(2x+m, 2y+n) denotes a pixel at coordinates Gᵢ(2x+m, 2y+n) of the skin image corresponding to the i^{th} layer of the downsampling pyramid; m denotes an offset in a horizontal direction, n denotes an offset in a vertical direction and ranges from −2 to 2, that is, a 5×5 pixel region around the central pixel is taken into account; and 2x, 2y: indicates scaling the coordinates in the image corresponding to the i^{th} layer of the downsampling pyramid to obtain pixels of the (i+1)th layer of the downsampling pyramid. By constructing the downsampling pyramid in this way, the resolution of the image may be effectively reduced, and major features of the image are maintained; during the downsampling process, weighted averaging is performed on 5×5 neighbors around the central pixel, such that the skin image is smoothened, and key structural information in the skin image is reserved, thus effectively recognizing tiny skin changes.

S220: an image upsampling pyramid is constructed, and the high-level skin image is restored to an original resolution. S220 specifically includes:

S221: scaling is performed based on pixel positions of a skin image obtained by downsampling to obtain pixel positions in the skin image corresponding to a target layer of the upsampling pyramid; and

S222: taking into account pixels within a preset variation range around the central pixel of the skin image corresponding to the target layer of the upsampling pyramid, weighted summation is performed to obtain pixels at corresponding pixel positions in the skin image corresponding to the target layer of the upsampling pyramid.

The upsampling pyramid is expressed as:

$${G}_{i}^{up} ( x , y ) = {\sum }_{m = - 2}^{2} {\sum }_{n = - 2}^{2} w ( m , n ) {G}_{i + 1} ( \frac{x}{2} + m , \frac{y}{2} + n ) \qquad ( 2 )$$

in formula (2),

$${G}_{i}^{up} ( x , y )$$

denotes a pixel at the coordinates (x, y) of the skin image corresponding to a (i+1)th layer of the upsampling pyramid. By means of the upsampling pyramid, the high-level skin image in the downsampling pyramid may be restored to the original resolution. By means of the upsampling pyramid, detailed information in the skin image may be maintained, and key structural information in the skin image may be reserved even in the resolution restoration process, thus further improving the detection accuracy of pressure injuries of skin.

S230: luminance signals of normal skin and luminance signals of skin with pressure injuries are extracted from the skin image obtained by upsampling, and the skin image preprocessed in S210 and S220 is magnified based on the Eulerian video magnification algorithm, wherein the YIQ value of each pixel in the skin image may be denoted by Y(x, y), I(x, y) and Q(x, y), a change of the luminance channel (Y) is magnified by the Eulerian video magnification algorithm to enhance tiny motions in the image; and Y(x, y) of a single-frame sequence at the position of each pixel in the skin image is extracted to obtain a luminance signal I(Y, t) of the pixel, which is expressed as:

$$I ( Y , t ) = Y ( x , y ) + δ ( t ) \frac{\partial Y ( x , y )}{\partial ( x , y )} \qquad ( 3 )$$

in formula (3), I(Y, t) denotes the luminance signal of the pixel (x, y) of the skin image obtained by upsampling at a time t; Y(x, y) denotes the luminance of the pixel (x, y); δ(t) denotes a luminance change at the time t, the luminance change is magnified, and the greater the luminance change, the better the amplification effect; and

$$\frac{\partial Y ( x , y )}{\partial ( x , y )}$$

denotes a change rate of the luminance with time. The luminance channel (Y) in the YIQ color space contains important luminance information, and by magnification, tiny changes related to pressure injuries of skin may be observed more clearly; and in the YIQ color space, by merely magnifying the change of the luminance channel (Y), a significant color distortion will not be introduced, which is of great importance for the detection of pressure injuries of skin and may maintain true color information of skin to prevent a mis-judgement. In conclusion, tiny motions in the skin image may be effectively enhanced by the Eulerian video magnification algorithm, which is of particular importance for improving the detection accuracy of pressure injuries of skin because the pressure injuries of skin are generally accompanied by tiny skin changes.

S230 further includes:

S231: frequency components related to skin with pressure injuries are separated out from the skin image obtained by downsampling by a band-pass filter according to a preset frequency. Specifically, the frequency components related to the skin with the pressure injuries are preferably blood and tissue motions in the skin and expressed as:

$${S}_{filtered ( t )} = H ( f ) \cdot S ( t ) \qquad ( 4 )$$

in formula (4), S(t) denotes an input signal (i.e., a luminance-time domain signal of a video stream formed by image frames), and with the time t as a horizontal axis and the luminance of pixels as a vertical axis, the input signal is a discrete one-dimensional array; S_{filtered}(t) denotes a signal subjected to band-pass filtering; H(ƒ) is the band-pass filter, wherein a low cut-off frequency and a high cut-off frequency of the band-pass filter are determined according to the frequency range of motions of a blood flow and tissue to be detected, and an ideal band-pass filter is adopted and expressed as:

$$H ( f ) = \{ \begin{matrix}\begin{matrix}1 & ( {D}_{1} \leq \sqrt{{u}^{2} + {v}^{2}} \leq {D}_{2} )\end{matrix} \\ 0\end{matrix} \qquad ( 5 )$$

in formula (5), D₁ and D₂ are the low cut-off frequency and the high cut-off frequency of the band-pass filter; \sqrt{u²+v²} denotes a distance in a frequency space; u denotes a horizontal frequency-domain component; and v denotes a vertical frequency-domain component. Specifically, the low cut-off frequency and the high cut-off frequency D₁ and D₂ of the band-pass filter are determined according to the frequency range of the motions of the blood flow and tissue to be detected, and the luminance signals are filtered by means of the band-pass filter to separate out signals within a specific frequency range; the ideal band-pass filter reserves frequency components between D₁ and D₂ and filters out other frequency components; and frequency components related to pressure injuries of skin, such as motions of the blood flow and tissue under the skin, may be effectively separated out by means of the band-pass filter. Pressure injuries of skin may be detected more accurately according to the frequency components separated out.

S232: spatial filtering and amplification are performed on signals output by the band-pass filter, the luminance signals of the normal skin and the luminance signals of the skin with the pressure injuries are extracted, and the signal subjected to band-pass filtering is processed by means of a spatial filter and expressed as:

$${S}_{spatial} ( x , y , t ) = IdealBPF ( x , y ) \cdot {S}_{filtered} ( t ) \qquad ( 6 )$$

in formula (6), Sₛₚₐₜᵢₐₗ(x, y, t) denotes the signal subjected to spatial filtering, IdealBPF(x, y) denotes the ideal band-pass filter, and S_{filtered}(t) denotes the signal subjected to band-pass filtering. In S232, spatial filtering and amplification are performed on the signals output by the band-pass filter, the luminance signals of the normal skin and the luminance signals of the skin with the pressure injuries are extracted, and tiny changes related to pressure injuries may be enhanced by spatial filtering and amplification, such that the pressure injuries of skin are observed more clearly, thus improving the detection accuracy of the pressure injuries of skin.

S300: correlation analysis is performed on the skin luminance signals to recognize a skin region with potential pressure injuries.

S300 specifically includes:

S310: Z-score standardization is performed on the luminance signal of the normal skin and the luminance signal of the skin with the pressure injuries. Specifically, for the luminance signals of the normal skin and the luminance signals of the skin with the pressure injuries, a mean of the luminance signals of the pixels are calculated and standardized to 0 to eliminate direct-current components from the signals, and the luminance signals subjected to Z-score standardization are expressed as:

$${L}_{zero \_ mean} ( x , y ) = L ( x , y ) - μ \qquad ( 7 )$$

in formula (7), μ is the mean of the luminance signals, L(x, y) denotes original luminance signals, and L_{zero_mean}(x, y) denotes the luminance signals subjected to Z-score standardization. In S310, the mean of the luminance signals is calculated and Z-score standardization is performed on the luminance signals to eliminate the direct-current components from the signals, such that subsequent correlation analysis for the detection of pressure injuries of skin may be simplified, thus improving the detection accuracy.

S320: covariance analysis is performed on the luminance signals of the normal skin and the luminance signals of the skin with the pressure injuries to obtain a correlation coefficient and a covariance of the luminance signals of the normal skin and the luminance signals of the skin with the pressure injuries. Because the time-domain signal input after processing in S200 is a discrete one-dimensional array, data need to be further processed to be used as reference indicators of the detection result. In this embodiment, the luminance signals of the normal skin and the luminance signals of the skin with the pressure injuries are denoted as X and Y respectively and may be obtained by processing in S200. Means X̅ and Y̅ and standard deviations σ_{X} and σ_{Y} of X and Y are calculated respectively. The covariance is calculated by:

$$cov ( X , Y ) = \frac{1}{n ′} {\sum }_{j = 1}^{n ′} ( {X}_{j} - \overline{X} ) ( {Y}_{j} - \overline{Y} ) \qquad ( 8 )$$

In formula (8), cov(X, Y) denotes the covariance, X and Y are the luminance signals of the normal skin and the luminance signals of the skin with the pressure injuries respectively, X̅ and Y̅ are the mean of the luminance signals of the normal skin and the means of the luminance signals of the skin with the pressure injuries respectively, n′ denotes the number of samples (i.e., the number of elements in the discrete one-dimensional array), Xⱼ denotes a j^{th} luminance signal of the normal skin, and Yⱼ denotes a j^{th} luminance signal of the skin with the pressure injuries.

σ_{X} and σ_{Y} are the standard deviation of the luminance signals of the normal skin and the standard deviation of the luminance signals of the skin with the pressure injuries, and ρ_{X,Y} is the correlation coefficient:

$${ρ}_{X , Y} = \frac{cov ( X , Y )}{{σ}_{X} {σ}_{Y}} \qquad ( 9 )$$

in formula (9), σ_{X} denotes the standard deviation of the luminance signals of the normal skin, σ_{Y} denotes the standard deviation of the luminance signals of the skin with the pressure injuries, and ρ_{X,Y} denotes the correlation coefficient.

In this embodiment, by calculating the correlation coefficient, the degree of linear relation between the luminance signals of the normal skin and the luminance signals of the skin with the pressure injuries may be evaluated. For example, if the correlation coefficient is close to 0, it indicates that there is no obvious linear relation between the two types of luminance signals, and potential pressure injuries may exit. By evaluating the correlation coefficient, a normal skin region and a skin region with potential pressure injuries may be distinguished, thus improving the detection accuracy of pressure injuries of skin; and by calculating the covariance, the difference between the luminance signals of the normal skin and the luminance signals of the skin with the pressure injuries may be quantified.

S330: the luminance signals of the normal skin and the luminance signals of the skin with the pressure injuries are normalized, that is, for the luminance signals X and Y of the normal skin and the skin with the pressure injuries, a maximum value and a minimum value are calculated respectively, and the luminance signals are normalized to [0-1]. By normalization, the luminance signals are unified in one range to facilitate subsequent analysis.

S340: differential analysis is performed on the normalized luminance signals of the normal skin and the normalized luminance signals of the skin with the pressure injuries. S340 specifically includes:

S341: a root-mean-square error of the luminance signals of the normal skin and a root-mean-square error of the luminance signals of the skin with the pressure injuries are calculated.

$$rms ( {X}^{′} ) = \sqrt{\frac{1}{n ′} {\sum }_{j = 1}^{n ′} {{X}^{′}}_{j}^{2}} \qquad ( 10 )$$

In formula (10), rms(X′) denotes the root-mean-square error of the normalized luminance signals, X′ denotes the normalized luminance signals, and n′ denotes the number of samples (i.e., the number of elements in the one-dimensional array).

S342: fast Fourier transform is performed on the normalized luminance signals of the normal skin and the normalized luminance signals of the skin with the pressure injuries, and signals processed by fast Fourier transform are extracted.

$${X}_{FFT} ( f ) = {\sum }_{t = 0}^{N - 1} X ( t ) {e}^{- j ′2π ft / N} \qquad ( 11 )$$

In formula (11), X_{FFT}(ƒ) indicates the signals processed by fast Fourier transform, X(t) denotes the amplitude of the luminance signals, and N denotes the length of the signals, ƒ denotes the frequency, and j′ denotes the imaginary unit. In S340, the root-mean-square errors of the normalized signals are calculated, and fast Fourier transform is performed on the normalized luminance signals, such that the difference between the luminance signals of the normal skin and the luminance signals of the skin with the pressure injuries is quantified; and specific frequency components related to pressure injuries of skin are recognized by frequency-domain analysis, such that the detection accuracy is improved.

As an optional technical solution, the method may further include: a step of extracting, based on empirical mode decomposition, intrinsic mode functions from the skin region with the potential pressure injuries. Specifically, empirical mode decomposition (EMD) analysis is performed on the luminance signals, and intrinsic mode (IMD) functions are extracted to recognize an intrinsic mode and features of the signals. This may disclose nonlinear characteristics and time-frequency characteristics of the signals to allow for a deep understanding of the dynamic change process of skin. An original signal is decomposed into a series of IMFs by EMD, and each IMF indicates an intrinsic oscillation mode of the signal and has the characteristics of instantaneous frequency and amplitude. For the detection of pressure injuries of skin, extraction of the IMFs means that the luminance signal is decomposed into a plurality of modes reflecting different physiological statuses of skin; the IMFs may disclose specific frequency modes or oscillating characteristics related to pressure sores, which are not obvious in correlation analysis, in the luminance signal, for example, an IMF may include specific frequency information of microcirculation disturbance or tissue damage of skin, and the information may be used for early recognition of the pressure sores.

S400, a power spectral density of the skin region with the potential pressure injuries and a power spectral density of the normal skin region are calculated respectively by means of a transfer function to obtain the detection result.

S400 specifically includes:

S410: the power spectral densities are calculated based on the signals processed by fast Fourier transform;

$${P}_{XX} ( f ) = {\vert {X}_{FFT} ( f ) \vert}^{2} \qquad ( 12 )$$

in formula (12), P_{XX}(ƒ) denotes the power spectral density, and X_{FFT}(ƒ) denotes the signals processed by fast Fourier transform; and

S420: the power spectral density of the normal skin region is divided by the power spectral density of the skin region with the potential pressure injuries to obtain a correlation graph, and the detection result is output;

$$H ( f ) = \frac{{P}_{XX , normal} ( f )}{{P}_{XX , injured} ( f )} \qquad ( 13 )$$

in formula (13), H(ƒ) denotes the transfer function, P_{XX,normal}(ƒ) denotes the power spectral density of the normal skin region, and P_{XX,injured}(ƒ) denotes the power spectral density of the skin region with the potential pressure injuries. In S400, the power spectral density of the skin region with the potential pressure injuries and the power spectral density of the normal skin region are calculated, and the ratio of the power spectral densities is calculated to obtain the detection result, which is used for evaluating the severity of the pressure injuries, and the level of the potential pressure injuries is output to help recognize feature frequencies related to the pressure injuries of skin, for example, if H(ƒ) changes significantly within some specific frequency ranges, these frequency ranges may be related to the pressure injuries, and the level of the potential pressure injuries may be evaluated according to the change of H(ƒ). Finally, the detection result, including a detected abnormal region, the level of the potential pressure injuries and the like, is displayed by the display allowing for interaction operation in the form of a graph and report, an alarm is given, and corresponding nursing suggestions are provided. The detection result may also be stored in a database to be inquired and compared with historical data by medical staff. All these functions provide a basis for early diagnosis and intervention in clinical application.

It should be noted that the sequential order of the above embodiments of the invention is merely for the purpose of description and does not indicate the superiority or inferiority of the embodiments. The process illustrated in the drawings may fulfil a desired outcome not definitely in the specific order illustrated or in a consecutive order. In some embodiments, multitasking and concurrent processing are available or may be favorable.

The embodiments in the specification are described progressively, similar portions in the embodiments may be cross-referenced, and in each embodiment, the differences from other embodiments are emphatically stated.

What is claimed is:

1. A method for non-invasive detection of pressure injuries of skin, comprising:
a step of extracting a skin video image and transforming the skin video image to a YIQ color space;
a step of magnifying the preprocessed skin video image based on a Eulerian video magnification algorithm to extract skin luminance signals;
a step of performing correlation analysis on the skin luminance signals to recognize a skin region with potential pressure injuries;
a step of normalizing luminance signals of normal skin and luminance signals of skin with pressure injuries;
a step of performing differential analysis on the normalized luminance signals of the normal skin and the normalized luminance signals of the skin with the pressure injuries, comprising:
a step of calculating a root-mean-square error of the luminance signals of the normal skin and a root-mean-square error of the luminance signals of the skin with the pressure injuries; and
a step of performing fast Fourier transform on the normalized luminance signals of the normal skin and the normalized luminance signals of the skin with the pressure injuries and extracting signals processed by fast Fourier transform;
a step of respectively calculating a power spectral density of the skin region with the potential pressure injuries and a power spectral density of a normal skin region to obtain a transfer function and outputting a detection result, comprising:
a step of calculating the power spectral densities based on the signals processed by fast Fourier transform; and
a step of dividing the power spectral density of the normal skin region by the power spectral density of the skin region with the potential pressure injuries to obtain a correlation graph and outputting the detection result, wherein the transfer function is expressed as:
$$H ( f ) = \frac{{P}_{XX , normal} ( f )}{{P}_{XX , injured} ( f )}$$
H(ƒ) denotes the transfer function, P_{XX,normal}(ƒ) denotes the power spectral density of the normal skin region, and P_{XX,injured}(ƒ) denotes the power spectral density of the skin region with the potential pressure injuries; by calculating the power spectral density of the skin region with the potential pressure injuries and the power spectral density of the normal skin region and calculating a ratio of the power spectral densities, the transfer function is obtained, and the detection result is output; and the detection result is used for evaluating the severity of the pressure injuries, and a level of the potential pressure injuries is output to recognize specific frequencies related to the pressure injuries of skin.

2. The method for non-invasive detection of pressure injuries of skin according to claim 1, wherein the step of magnifying the preprocessed skin video image based on a Eulerian video magnification algorithm to extract skin luminance signals comprises:
a step of constructing an image downsampling pyramid and extracting a high-level skin image by weighted averaging of adjacent pixels;
a step of constructing an image upsampling pyramid and restoring the high-level skin image to an original resolution; and
a step of extracting the luminance signals of the normal skin and the luminance signals of the skin with the pressure injuries from a skin image obtained by upsampling.

3. The method for non-invasive detection of pressure injuries of skin according to claim 2, wherein the step of constructing an image downsampling pyramid and extracting a high-level skin image by weighted averaging of adjacent pixels comprises:
a step of selecting coordinates of a central pixel of the skin image corresponding to a target layer of the downsampling pyramid and scaling the coordinates to obtain a corresponding pixel position in the skin image corresponding to a layer below the target layer of the downsampling pyramid; and
a step of taking into account pixels within a preset variation range around the central pixel of the skin image corresponding to the target layer, performing weighted summation to obtain pixels at corresponding pixel positions in the skin image corresponding to the layer below the target layer.

4. The method for non-invasive detection of pressure injuries of skin according to claim 3, wherein the step of constructing an image upsampling pyramid and restoring the high-level skin image to an original resolution comprises:
a step of performing scaling based on pixel positions of a skin image obtained by downsampling to obtain pixel positions in the skin image corresponding to a target layer of the upsampling pyramid; and
a step of taking into account pixels within a preset variation range around the central pixel of the skin image corresponding to the target layer of the upsampling pyramid, performing weighted summation to obtain pixels at the corresponding pixel positions in the skin image corresponding to the target layer of the upsampling pyramid.

5. The method for non-invasive detection of pressure injuries of skin according to claim 4, wherein the step of magnifying the preprocessed skin video image based on a Eulerian video magnification algorithm to extract skin luminance signals further comprises:
a step of separating out, by a band-pass filter, frequency components related to the skin with the pressure injuries from the skin image obtained by downsampling according to a preset frequency; and
a step of performing spatial filtering and amplification on signals output by the band-pass filter and extracting the luminance signals of the normal skin and the luminance signals of the skin with the pressure injuries.

6. The method for non-invasive detection of pressure injuries of skin according to claim 1, wherein the step of performing correlation analysis on the skin luminance signals to recognize a skin region with potential pressure injuries comprises:
a step of performing Z-score standardization on the luminance signals of the normal skin and the luminance signals of the skin with the pressure injuries; and
a step of performing covariance analysis on the luminance signals of the normal skin and the luminance signals of the skin with the pressure injuries to obtain a correlation coefficient and a covariance of the luminance signals of the normal skin and the luminance signals of the skin with the pressure injuries.

7. The method for non-invasive detection of pressure injuries of skin according to claim 1, further comprising a step of extracting, based on empirical mode decomposition, intrinsic mode functions from the skin region with the potential pressure injuries.
